(19) United States
(12) Patent Application Publication
(10) Pub. No.: US 2013/0337070 A1
(43) Pub. Date: Dec. 19, 2013

(54) Coated Nanoparticle Therapy for Skin Cancer

(71) Applicants: Peter Brenneisen, Cologne (DE); Sudipta Seal, Orlando, FL (US)

(72) Inventors: Peter Brenneisen, Cologne (DE); Sudipta Seal, Orlando, FL (US)

(73) Assignee: University of Central Florida Research Foundation Inc., Orlando, FL (US)

(21) Appl. No.: 13/903,931

(22) Filed: May 28, 2013

Related U.S. Application Data
(60) Provisional application No. 61/651,818, filed on May 25, 2012.

(51) Int. Cl. A61K 9/14; A61K 31/721; A61K 33/24

(52) U.S. Cl. CPC A61K 9/146; A61K 33/24; A61K 31/721; Y10S 977/773; Y10S 977/915; B82Y 5/00
USPC 424/493; 424/617; 977/773; 977/915

(57) ABSTRACT

Disclosed herein are compositions useful as therapy and prevention of skin neoplasms, namely melanoma. Also disclosed herein are methods of using polymer-coated cerium nanoparticles as therapy against skin cancer. Further, disclosed are combination therapies involving polymer-coated cerium nanoparticles with other anti-neoplastic agents.

### INTRODUCTION

Reactive oxygen species (ROS) are by-products of cellular metabolism, generated primarily by mitochondrial activity, other endogenous (52, 53) or exogenous sources such as xenobiotics (25), cytostatics (49, 65) as well as UV-radiation (9). A variety of soluble factors stimulate the generation of ROS, which via alterations of the intracellular redox state and/or oxidative modification of proteins exert their action on signaling components (59, 60). If not regulated properly, by e.g. antioxidants, excess ROS results in oxidative stress, thus damaging cellular macromolecules and inhibiting cellular functions which may resulting in some pathologies including cancer (47, 63).

The number of malignant melanoma being the most aggressive type of skin cancer is rapidly increasing, suggesting a doubling of the incidence every 10-20 years (18, 24). Although surgical treatment of early melanoma leads to high cure rates, the prognosis of 5-year survival for advanced melanoma is rather poor (4) as a chronic increased ROS level favours survival and proliferation (16, 64). Those melanoma tend to metastasize and to show some resistance to classical treatments (7). This reflects the current lack of therapeutic approaches for treating advanced melanoma (19). In addition, epidemiological studies showed an increased risk of secondary cancers in individuals having a history of cutaneous melanoma (35). These facts pose a great challenge for finding new approaches for the chemoprevention of the progression of that type of cancer. Breaking ROS tolerance of melanoma cells by either impairing their antioxidant system or further elevating their intracellular ROS level by new therapeutics might hold a future promise as an alternative therapeutical approach.

Nanomedicine, the medical application of nanotechnology, deals with the application of structures <100 nanometers cancer therapy (15) in the near future. A nanoparticle-based therapy may have the potential as supplemental therapy supporting the classical anticancer strategies. If future studies show that a nanoparticle-based anticancer therapy is as effective as established therapies and has less side effects, the application of nanoparticles as major anticancer approach is conceivable. In earlier studies, vacancy engineered cerium oxide based nanoparticles exhibited superoxide dismutase and catalase mimetic activity in a cell free system (27, 44).

### BRIEF DESCRIPTION OF THE DRAWINGS

FIG. 1 Effect of coated versus uncoated cerium oxide nanoparticles in vitro. A Dextran-coated nanoparticles lower aSMA expression. Subconfluent human dermal fibroblasts (HDF) were either mock-treated or pretreated for 48 h with 150 μm coated or uncoated CNP before addition of rTGFβ1. TGFβ1 and the cerium oxide particles were present for an additional 48 h α-tubulin was used as loading control. Three independent experiments were performed B Coated and uncoated nanoparticles lower cell viability of melanoma cells. Subconfluent melanoma cells (A375) were treated with coated or with uncoated cerium oxide nanoparticles or untreated (ct). The percentage of living cells after 96 h was measured by MTT assay. The experiments were performed in two independent experiments with triplicates in each group.

FIG. 2 Inhibitory effect of CNP on the growth of A375 xenografted mice. A Images of dissected tumors. Three groups of mice were performed. Group 1 is vehicle treated (mock-treated, (−)), group 2 is CNP treated starting with day 1 after tumor injection and group 3 is CNP treated starting with day 10 after tumor injection. After 30 days the tumors were dissected. B Tumor volume. The tumor volume of 6 mice per group was measured. **P<0.01 versus ct (ANOVA, Dunnett's test). Data are presented as means ±s.d.. C Tumor weight. The tumor weight of 6 mice per group was measured. **P<0.01 versus ct (ANOVA, Dunnett's test). Data are presented as means ±s.d.. D H&E and CD31 staining. Formalin fixed tissues were embedded in paraffin and sectioned for immunohistological analysis of angiogenesis via CD31 staining and H&E staining for morphology. The percentage of CD31 staining is presented. ***P<0.001 versus ct (ANOVA, Dunnett's test). Data are presented as means ±s.e.m..

FIG. 3 A Cytotoxicity of CeO₂ on normal and melanoma cells. Subconfluent melanoma cells (A375), human dermal fibroblasts (HDF) and human endothelial cells (HMEC-1) were treated with CNP or with dextran only (mock-treated) or untreated (ct). The percentage of living cells after 96 h was measured by MTT assay. The experiments were performed in two independent experiments with triplicates in each group. *P<0.05 versus ct (Student t test). B CC50 value against melanoma cells. Subconfluent melanoma cells (A375) were treated with various concentrations of CNP. The percentage of living cells after 96 h was measured by MTT assay. The experiments were performed in three independent experiments. C Anti-invasive impact of CNP on melanoma cells. Subconfluent melanoma cells (A375) were treated with CNP or mock-treated before used for invasion assays. The invasive capacity of these cells was tested with conditioned media of HDF (CMHDF) and myofibroblasts (CMMF). The data represent the mean ±s.e.m. of three independent experiments. *P<0.05 versus CMMF (Student t test).

FIG. 4 Increased ROS level and oxidative damage in CeO₂-treated tumor cells. A Intracellular generation of ROS by CNP. Subconfluent A375 and HDF were serum starved for 24 h. The cells were incubated with DCF for 30 min and than either mock-treated, CNP or doxorubicin treated. The increase in DCF fluorescence as a measure of increase in ROS was followed over 90 min. Results are representative for two independent experiments with triplicates in each group. CM, conditioned medium. B Prooxidant effect of CNP. Hydrogen peroxide was detected using the Amplex Red reagent. The data represent the mean±s.e.m. of three independent experiments. *P<0.05 versus ct (ANOVA, Dunnett's test). C Detection of CNP-induced carbonylated proteins in vitro and in vivo. A375 cells were exposed to CNP for different time intervals before oxidized proteins were detected by western blot analysis via derivatisation with 2,4-dinitrophenyl hydrazine. H₂O₂ was used as positive control and a-tubulin as loading control (a). Three independent experiments were performed. Protein lysates prepared from flash frozen tissues of the xenograft mouse model were used in Western blot procedures to assess generalized protein oxidation (b). D. Profiling sulfenic acid modifications in A375 cells. Protein was isolated from cells incubated in media containing dimedone, in combination with H₂O₂ for 2 h or CNP for 24 h. Sulfenic acid modified proteins were analyzed by Western blot with the α-hapten antibody. α-tubulin was used as loading control. Three independent experiments were performed. E. CNP treatment leads to ROS-dependent accumulation of HIF1α. A375 cells were treated with CNP for 24 h. CoCl2, a hydroxylase inhibitor, was added for the last 4 h. The amount of HIF1α protein was determined by western blot analysis. a-tubulin was used as loading control. Three independent experiments were performed.

FIG. 5 CNP treatment caused apoptosis in A375 cells. A Subcellular distribution of cytochrome c. Subconfluent A375 cells were either mock-treated or incubated for 12 h and 24 h CNP and cytochrome c level was determined by western blot in the cytosolic fraction. H₂O₂ was used as positive control and a-tubulin as a loading control. Three independent experiments were performed. B Increased Caspase-3 activity in melanoma cells. Caspase-3 activity was measured using a fluorometric caspase-specific substrate (Ac-DEVD-AMC). Staurosporine was used as positive control. Results represent the means of three separate experiments, and error bars represent the standard error of the mean. Statistical significance was shown by the Student's t-test versus ct (*P<0.05). C Cleavage of Poly(ADP-ribose)polymerase (PARP). Representative Western blot shows PARP cleavage after incubation of melanoma cells for 24 h with CNP compared to mock-treated control (ct). H₂O₂ was used as positive control and α-tubulin as loading control. Three independent experiments were performed.

FIG. 6 Expression of Caveolin-1 in melanoma cells. Representative Western blot demonstrates caveolin-1 protein expression in A375 cells either mock-treated, CNP treated for 24 h or preincubated with apocynin for 1 h or N-acetyl-L-cystein for 4 h and than CNP treated for 24 h. H₂O₂ was used as a positive control and a-tubulin as a loading control. Three independent experiments were performed.

Submitted herewith is Appendix A, which sets forth supplemental data including additional figures and figure legends which are referenced herein. The figures of the Appendix are referenced with an “s” which refers to ‘supplemental’. The Appendix includes figures S1-S5.

### DETAILED DISCUSSION

As both the incidence of melanoma is increasing faster than that of other cancers and the chemotherapeutical treatment of a majority of patients with metastatic melanoma often results in adverse reactions and response rates which are not high enough to significantly affect median survival, novel therapeutical approaches must be the objective for the near future. In this disclosure, it has been shown for the first time, in vitro and in vivo, that concentrations of polymer-coated cerium oxide nanoparticles (CNPs) being non-toxic for stromal cells exhibit a direct reactive oxygen species-dependent cytotoxic (proapoptotic) and anti-invasive effect on melanoma cells. Accordingly, embodiments of the present disclosure relate to novel therapies for melanoma and other cancers.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

According to one embodiment, provided is a composition for treating skin cancer including polymer-coated cerium oxide nanoparticles. The nanoparticles typically possess a coating that selectively produces oxidative stress in skin cancer cells. In a more specific embodiment, the nanoparticles are of a size between 1-100 nm, 1-50 nm, 1-30 nm, 10-30 nm, or 15-25 nm.

In certain embodiments, the CNPs have a predominant +4 to 3+ ratio of the oxidation states on their surface. As used herein, a predominant ratio of oxidation states means a higher percentage of one oxidation state (or valence state) compared to another.

The polymer coating on the cerium nanoparticles may include polyhydroxylated polymers such as natural polymers or hydroxyl-containing polymers including, but not limited to, multiply-hydroxylated polymers, polysaccharides, carbohydrates, polyols, polyvinyl alcohol, poly amino acids such as polyserine, or other polymers such as 2-(hydroxyethyl)methacrylate, or combinations thereof. In a specific embodiment, the polyhydroxylated polymers are polysaccharides. In a more specific embodiment, the polysaccharides include but are not limited to dextran, mannan, pullulan, maltodextrin, starches, cellulose and cellulose derivatives, gums, xanthan gum, locust bean gum, or pectin, combinations thereof. In an even more specific embodiment, the polymer is dextran or mannan.

While not being bound to any mechanism or mode of action, it is believed that nanoparticles taught herein selectively increase H₂O₂ in skin cancer cells compared to non-cancer cells.

According to another embodiment, provided is a method of treating skin cancer in a subject in need. The method includes administering to the subject a therapeutically effective amount of a composition comprising polymer-coated cerium oxide nanoparticles. A subject in need is one who has skin cancer cells or a family history of skin cancer. Thus, according to the present disclosure, compositions taught herein may be administered to one diagnosed with skin cancer or one who is at a higher risk of getting skin cancer. In a specific example, caucasian subjects lacking dark brown eyes are treated prophylactically with compositions taught herein.

The terms “skin cancer” and “skin neoplasms” are used herein interchangeably. Examples of skin cancers include, but are not limited to, melanoma, basal cell cancer, and squamous cell cancer.

In the methods of the present disclosure, the amount of CNP administered to a subject is sufficient to achieve a desired effect or treatment. The amount administered may vary depending on the goal of the administration, the health and physical condition of the subject being treated, the subject's age, the degree of resolution desired, the formulation and/or activity of the subject composition, the treating clinician's assessment of the medical situation, the body weight/mass of the subject, as well as the severity of the disease or condition being treated, among other relevant factors. The dosage may also be determined based on the existence, nature, extent, and severity of any adverse side-effects that might accompany the administration of a particular composition.

The term “therapeutically effective amount” intends to describe concentrations or amounts of CNPs according to the present invention which are therapeutically effective in treating neoplasm (I.e. tumors, cancers, etc.), pre-neoplasm, proliferative disorders, and/or precancerous lesions or the various conditions or disease states including hyperproliferative cell growth.

The term “effective amount” shall mean an amount or concentration of a compound or composition according to the present invention which is effective within the context of its administration, which may be inhibitory, prophylactic and/or therapeutic. Compounds according to the present invention are particularly useful for providing favorable change in the disease or condition treated, whether that change is a remission, a decrease in growth or size of cancer or a tumor or other effect of the condition or disease to be treated, a favorable physiological result or a reduction in symptomology associated with the disease or condition treated.

It is expected that the dosage will fall in a relatively broad range that can be determined through routine trials. For example, in some embodiments, the dosage is not more than an amount that could be otherwise irreversibly toxic to the subject (i.e., the maximum tolerated dose). In other cases, the dosage is near or even well below the toxic threshold, but is still an effective amount to treat the target disease or condition. In some embodiments, the dosage ranges from 0.5 mg/kg/day or more, up to 5 mg/kg/day or more, up to 10 mg/kg/day or more, up to 12.5 mg/kg/day or more, up to 15 mg/kg/day or more, up to 1 7.5 mg/kg/day or more, up to 20 mg/kg/day or more, up to 25 mg/kg/day or more, or up to 30 mg/kg/day.

In practicing the methods of the present disclosure, the CNPs may be administered to the subject according to any convenient administration protocol. As such, one or more of the CNPs disclosed herein may be administered to a subject via a suitable route of administration and in a sufficient amount to effectively treat a skin neoplasm. The subject methods are generally used to establish and/or maintain a target concentration of one or more of the subject CNPs in the tissues of a subject in order to treat the skin neoplasm. Administration routes of interest include, but are not limited to, topical, transdermal, enteral (e.g., through the gastrointestinal tract of the subject, including oral and rectal administration), and parenteral routes (e.g., intravenous injection, intra-arterial injection, intramuscular injection, or intraosseous infusion). While any convenient administration route may be employed, two administration routes of interest include transdermal and enteral.

Pharmaceutical compositions comprising the active compounds (e.g., CNPs) of the invention may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active compounds into preparations which can be used pharmaceutically. The actual pharmaceutical composition administered will depend upon the mode of administration. Virtually any mode of administration may be used, including, for example topical, oral, systemic, inhalation, injection, transdermal, etc.

The active compound may be formulated in the pharmaceutical compositions per se, or in the form of a pharmaceutically acceptable salt. As used herein, the expression “pharmaceutically acceptable salt” means those salts which retain substantially the biological effectiveness and properties of the active compound and which is not biologically or otherwise undesirable. Such salts may be prepared from inorganic and organic acids and bases, as is well-known in the art. Typically, such salts are more soluble in aqueous solutions than the corresponding free acids and bases.

For topical administration, the active compound(s) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example,tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration, the compositions may take the form of tablets, chewing gum or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For administration by inhalation, the active compound(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For prolonged delivery, the active compound(s) can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives; e.g., as a sparingly soluble salt.

Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver active compounds(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

In some embodiments, a transdermal composition may be used to administer the CNPs to a subject, i.e., the CNPs is transdermal̂ administered to the subject. In such instances, a transdermal formulation which includes the CNPs is contacted with a convenient topical site (e.g., skin site) of the subject. Topical sites of interest include both mucosal sites and keratinized skin sites, and therefore include, but are not limited to: mouth, nose, eyes, rectum, vagina, arms, leg, torso, head, etc. The surface area that is covered by the composition following application is sufficient to provide for the desired amount of agent administration, and in some embodiments ranges from 10 cm² to 200 cm², such as from 20 cm² to 150 cm², and including from 40 cm² to 140 cm², e.g., 100 cm².

In practicing methods of the invention, any convenient transdermal composition may be employed. Transdermal compositions, also known as transdermal patches or skin patches, are adhesive patches containing an active agent, where the compositions are configured to be placed on the skin to deliver the active agent through the skin. Transdermal patches deliver the active agent by percutaneous absorption, which is the absorption of substances through unbroken skin. After a transdermal patch is applied to the skin, the active agent contained in the patch passes through, or permeates the skin and can reach its site of action through systemic blood flow. Alternatively, the transdermal patch may be placed on the desired treatment site such that the medication contained in the patch is delivered topically.

Transdermal compositions may be formulated to provide for multi-day delivery of a therapeutically effective amount of a CNPs to a subject. By “multi-day delivery” is meant that the composition is formulated to provide a therapeutically effective amount of a CNPs to the subject when the composition is applied to a skin site of the subject for a period of time that is, e.g., 1 day or longer, such as 2 days or longer, e.g., 3 days or longer, such as 5 days or longer, including 7 days or longer, such as 10 days or longer.

In some embodiments, the compositions may provide delivery of a target dosage of active agent ranging from, e.g., 0.5 mg/kg/day or greater over a desired period of time (e.g., 1 week), including 1 .0 mg/kg/day or greater, such as 5 mg/kg/day, 10 mg/kg/day, 12.5 mg/kg/day, 15 mg/kg/day, 17.5 mg/kg/day, 20 mg/kg/day, 25 mg/kg/day, 30 mg/kg/day, or greater over a desired time period.

The size (i.e., area) of the transdermal compositions may vary. In certain embodiments, the size of the composition is chosen in view of the transdermal flux rate of the CNPs and the desired dosage to be achieved. For example, if the transdermal flux rate is 5 μg/cm²/hr and the target dosage is 5 mg/day, then the transdermal composition is chosen to have an area of about 42 cm². Or for example, if the transdermal flux is 5 μg/cm²/hr and the target dosage is 10 mg/day, then the transdermal patch is chosen have an area of about 84 cm². In certain embodiments, the compositions have dimensions chosen to cover an area of skin when applied to a skin site that ranges from 10 cm² to 200 cm², such as 20 cm² to 150 cm², including 40 cm² to 140 cm².

In some instances, transdermal compositions of the invention include a matrix layer which contains a active agent, and optionally a backing layer and/or release liner. The matrix may include a pressure sensitive adhesive. The terms “pressure sensitive adhesive”, “self adhesive”, and “self stick adhesive” mean an adhesive that forms a bond when pressure is applied to adhere the adhesive with a surface. In some instances, the adhesive is one in which no solvent, water, or heat is needed to activate the adhesive. For pressure sensitive adhesives, the degree of bond strength is proportional to the amount of pressure that is used to apply the adhesive to the surface. Pressure sensitive adhesives of interest include, but are not limited acrylate polymers, such as acrylate copolymers, including carboxylated acrylate polymers and copolymers. Acrylate copolymers of interest include copolymers of various monomers which may be “soft” monomers, “hard” monomers, and optionally “functional” monomers. Also of interest are blends including such copolymers. The acrylate copolymers can be composed of a copolymer including bipolymer (i.e., made with two monomers), a terpolymer (i.e., made with three monomers), or a tetrapolymer (i.e., made with four monomers), or copolymers made from even greater numbers of monomers. The acrylate copolymers can include cross-linked and non-cross-linked polymers. The polymers can be cross-linked by known methods to provide the desired polymers.

The matrix as described herein may contain a percutaneous absorption enhancer. The percutaneous absorption enhancer may facilitate the absorption of the active agent by the skin of the subject. The percutaneous absorption enhancer may also be referred to as a percutaneous permeation enhancer because it may facilitate not only the percutaneous absorption of the active agent, but also the percutaneous permeation of the active agent through the skin of the subject.

As summarized above, transdermal compositions of interest may include a backing (i.e., support layer). The backing may be flexible to an extent that it can be brought into close contact with a desired topical location of a subject. The backing may be fabricated from a material that it does not absorb the active agent, and does not allow the active agent to be released from the side of the support. The backing may include, but is not limited to, non-woven fabrics, woven fabrics, films (including sheets), porous bodies, foamed bodies, paper, composite materials obtained by laminating a film on a non-woven fabric or fabric, and combinations thereof.

In some embodiments, a release liner is provided on the active agent layer (i.e., matrix), and specifically on a surface of the active agent layer that is distal (i.e. opposite) from the backing layer, if present. The release liner facilitates the protection of the active agent layer. The release liner may be prepared by treating one side of polyethylene-coated wood free paper, polyolefin-coated glassine paper, a polyethylene terephthalate (polyester) film, a polypropylene film, or the like with a silicone treatment.

Transdermal compositions of interest for use methods of the invention include those described in U.S. patent application Ser. No. 13/052,955, filed on Mar. 21, 201 1 ; and U.S. patent application No. 61 /467,337, filed on Mar. 24, 201 1; the entire disclosures of which are hereby incorporated by reference. Other transdermal formulations of interest include, but are not limited to: those described in U.S. Pat. Nos. 7,638,140; 7,220,473; 7,150,881; 7,070,808; 6,929,801; 6,689,379; 6,638,528; 6,630,514; and 5,786,390; the disclosures of the transdermal formulations described therein being incorporated herein by reference.

Following application, a transdermal composition may be maintained at the topical site to which it has been applied for a desired amount of time, e.g., to deliver a desired amount of a CNPs to the subject. In some embodiments, the period of time that the composition is maintained at the site of application is 1 hour or longer, such as 5 hours or longer, including 10 hours or longer, such as 24 hours or longer, such as 48 hours or longer, e.g., 72 hours or longer, such as 96 hours or longer, such 168 hours or longer, such as 240 hours or longer.

After the transdermal composition has been applied to the skin site for a desired amount of time (i.e., an amount of time sufficient to deliver a target dose of the CNPs to the subject over a period of time), the composition may be removed from the skin site. A new transdermal composition may be applied at the same or at a different site. The new transdermal composition may be applied to a different skin site to reduce the possible occurrence of skin irritation and/or skin sensitization at the prior site of application. In practicing methods described herein, a single transdermal composition may be applied once, or multiple transdermal compositions may be applied repeatedly over a specified period of time period, e.g., the course of the disease condition being treated, where the dosing schedule when a plurality of compositions are administered over a given time period may be daily, weekly, biweekly, monthly, etc.

For enteral administration, any convenient type of formulation suitable for enteral, e.g., oral or rectal, administration may be employed. In some embodiments, a suitably-formulated composition of the present disclosure (e.g., a pill) may be orally administered to a subject to treat a skin neoplasm. The composition may be administered to the subject repeatedly over a desired period of time (e.g., twice a day for 7 days) in order to establish and/or maintain a desired concentration of the CNPs in the subject that effectively treats the skin neoplasm.

CNPs of the present disclosure may be formulated for enteral administration through the gastrointestinal tract of the subject, including oral and rectal administration. Oral administration can be accomplished using, e.g., solid formulations, including pills, tablets, capsules, and the like, or, e.g., liquid formulations, including solutions, suspensions, emulsions, syrups, elixirs, and the like. Rectal administration can be accomplished using, e.g., ointments, suppositories, enemas, and the like.

For oral preparations, the CNPs can be used alone or in combination with appropriate additives to make tablets, powders, granules, or capsules, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and, if desired, with diluents, buffering agents, moistening agents, preservatives, and/or flavoring agents.

Furthermore, the CNPs can be made into suppositories by mixing with a variety of bases such as emulsifying bases or water-soluble bases. The CNPs of the present invention can be administered rectally via a suppository. The suppository can include vehicles such as cocoa butter, carbowaxes, and polyethylene glycols, which melt at body temperature, yet are solidified at room temperature.

Unit dosage forms for oral or rectal administration, such as syrups, elixirs, and suspensions may be provided wherein each dosage unit, for example, teaspoonful, tablespoonful, tablet or suppository, contains a predetermined amount of the CNPs of the present disclosure. Similarly, unit dosage forms for injection or intravenous administration may comprise one or more CNPs in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of CNPs of the present invention calculated in an amount sufficient to produce the desired effect in association with a pharmaceutically acceptable diluent, carrier or vehicle. The specifications for the unit dosage forms of the present disclosure depend on the particular compound or CNPs employed and the effect to be achieved, and the pharmacodynamics associated with each compound in the subject.

In practicing the methods of the present disclosure, an effective amount of a suitably-formulated enteral composition described herein may be administered to a subject via any desired route of administration. Such administration may be a single administration, or may be repeated one or more times in order to establish and/or maintain a desired concentration of the compound in a target tissue of a subject.

In some instances, the methods may include administering CNPs to a subject in conjunction with one or more additional therapies to treat a skin neoplasm, i.e., one or more additional skin neoplasm active agents. As such, the compositions of the present disclosure may be used alone to treat a skin neoplasm, or alternatively, for example, they may be used in combination with or as an adjunct to conventional treatment with other medications, e.g., anti-neoplastic agents. The compositions and methods of the present disclosure may generally be used in combination with any anti-neoplastic agents, such as conventional and/or experimental chemotherapeutic agents (i.e., anti-neoplastic agents), radiation treatments, and the like. The conjunctive therapy may be co-administered with the CNPs. The term co-administered, when used, for example with respect to CNP and an antineoplastic agent, refers to administration of the agents such that both can simultaneously achieve a physiological effect. The agents, however, need not be administered together. In certain embodiments, administration of one can precede administration of the other, however, such co-administering typically results in the agents being simultaneously present in or on the body.

Anti-neoplastic agents that may be used in combination with the CNPs and methods of the present disclosure include, but are not limited to, e.g., alkylating agents and platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab, kinase inhibitors (e.g., imatinib, erlotinib, gefitinib, plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins, and the like. In certain embodiments, the subject methods may include a diagnostic step. Individuals may be diagnosed as being in need of the subject methods using any convenient protocol suitable for use in diagnosing the presence of a skin neoplasm, such as visual diagnosis, biopsy, dermatoscopy, etc. In addition, individuals may be known to be in need of the subject methods, e.g., they are suffering from a skin neoplasm. Methods of the present disclosure may further include assessing the efficacy of the treatment protocol, which may be performed using any convenient protocol, e.g., by monitoring the rate of regression and/or progression of the skin neoplasm conditions (such as by using the diagnosis protocols, e.g., as described above).

Methods of the invention are suitable for use with a variety of different subjects. Subjects of interest include, but are not limited to: mammals, both human and non-human, including the orders carnivore (e.g., dogs and cats), rodentia (e.g., mice, guinea pigs, and rats), lagomorpha (e.g. rabbits) and primates (e.g., humans, chimpanzees, and monkeys). In some embodiments, the subjects, e.g., patients, are humans.

### EXAMPLES

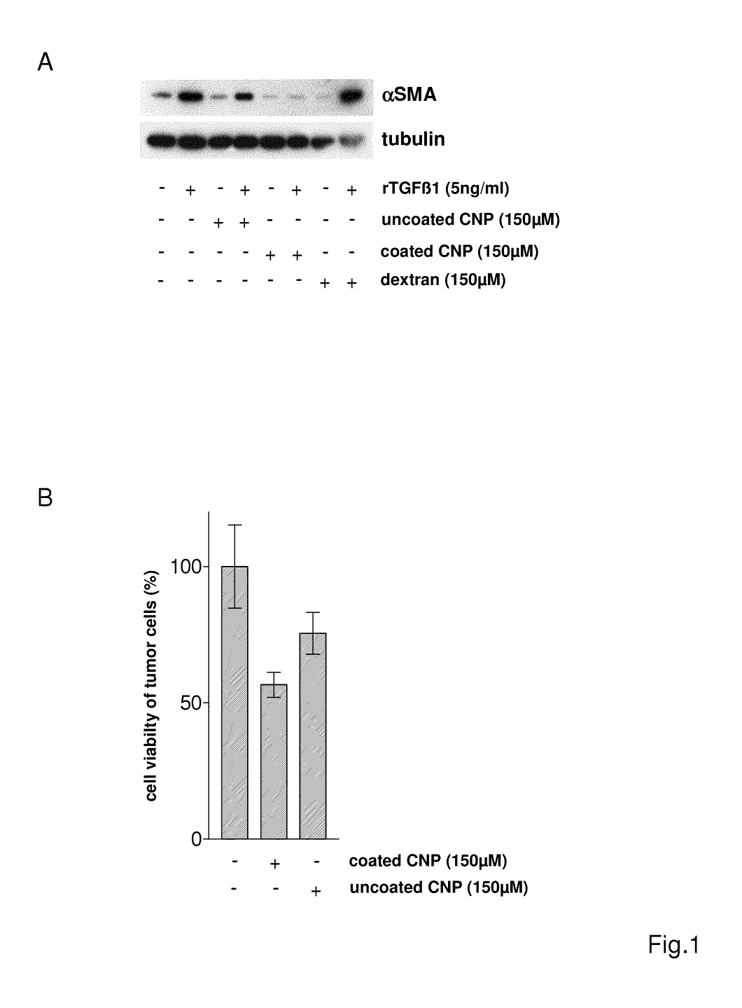

In order to decide on the use of uncoated or dextran-coated cerium oxide nanoparticles (CNP) as a potential therapeutical tool to lower tumor invasion and metastasis in vitro and in vivo, the effect of both types of these nanoparticles on the expression of α-smooth muscle actin, a biomarker of myofibroblastic cells promoting tumor invasion (13), and on the viability of tumor cells was tested. TGFβ1 significantly increased the amount of the αSMA protein compared to the untreated controls. That TGFβ1-mediated αSMA expression could completely be abrogated by dextran-coated CNP, but not uncoated CNP. The polymer dextran had no effect on αSMA expression (FIG. 1A). These data indicate the use of coated CNP to prevent a myfibroblast dependent increase in tumor invasion. Furthermore, the treatment of melanoma cells with coated or uncoated cerium oxide nanoparticles increased cell death of the tumor cells. However, it is interesting that the coated CNP showed slightly stronger effect in context of cell toxicity (FIG. 1B). In conclusion, the dextrancoated nanoparticles showed a more promising effect regarding the tested parameters.

Therefore, for the further experiments this study focussed on the importance of polymer-coated cerium oxide nanoparticles as potential anticancer agent for prevention of invasion of tumor cells in vivo and in vitro. The cytotoxicity and oxidative stress caused by 5-nm cerium oxide (CeO₂) nanoparticles in cultured human melanoma cells (A375) was investigated. Concentrations of CNP being non-toxic on stromal cells (e.g. fibroblasts, endothelial cells) showed significantly decreased cell viability in tumor cells as a function of nanoparticle concentration and exposure time. Indicators of oxidative stress and apoptosis, including total reactive oxygen species, carbonylated proteins, PARP cleavage and caspase-3 activity were quantitatively assessed. It is concluded from the data that free oxygen radicals generated by CeO₂ nanoparticles produce significant oxidative stress in tumor cells resulting in a decrease in cell viability and lowering of the invasive capacity of cancer cells.

### 1. Characterization and Cellular Uptake of CNP

The characteristics of nanoparticles such as size and shape are crucial for determining their functional role in tumor cells. Therefore, the synthesized particles were examined using high-resolution transmission electron microscopy (HRTEM) to determine particle size (FIG. S1A (a)). The micrographs show an individual particle size of ˜5 nm and depicting lattice fringes of CNP with 0.31 nm SAED pattern showing fluorite structure (FIG. S1A (b)).

Both, the coated and non-coated samples have a mixed valance state of Ce³⁺ and Ce⁴⁺ on the surface with a stable predominance of 4+ oxidation state for coated samples (ratio Ce³⁺/Ce⁴⁺=21/79) and a predominance of 3+ for non-coated samples (ratio Ce³⁺/Ce⁴⁺=67/33) (FIG. S1B) measured and calculated by X-ray spectroscopy. As for the proposed SOD mimetic activity of CNP (31) a higher percentage of Ce3+is needed. For the subordinated catalase mimetic activity (3, 44) the lower percentage of Ce³⁺ of the coated particles is sufficient.

In addition, a slow reduction of freshly synthesized non-coated CNP upon aging in water for 7 days was confirmed earlier (3), reflecting a change in the ratio of Ce⁴⁺/Ce³⁺ towards Ce³⁺. The aging of nanoparticles in dextran does not reduce CNP to the same extent as the non-coated CNP post 7 days of aging, indicating a stable Ce⁴⁺/Ce³⁺ ratio towards Ce4+ at neutral pH.

Moreover, the dextran-coated nanoparticles have an added advantage of stability in alkaline to neutral medium in contrast to non-coated particles which are stable in acidic media only. Another significant advantage by coating these particles with dextran is that the individual nanoparticles are of the size 3-5 nm and do not agglomerate in suspension (FIG. S4A). High resolution transmission electron microscopy indicated no agglomeration tendency over a period of 3 months (data not shown). Taken together, the stable dextran-coated cerium oxide nanoparticles were used for further studies.

Human melanoma cell line (A375) was used to demonstrate a cellular uptake of the particles. Cells were treated with fluorescein-isothiocyanate (FITC)-labeled CNP for 4 h and, thereafter, prepared for visualization. The CNP were ubiquitously distributed in the cytosol (FIG. S1C (b)) compared with mock-treated control (a). To confirm the presence of nanoceria in A375 cells, transmission electron microscopy (TEM) was performed as well. The TEM micrographs of human melanoma cells show CeO₂ nanoparticles at 1 h upon treatment as solid black dots localized in the cytosol (FIG. S1C (d)).

2. Effect of CNP on Tumor Growth and Angiogenesis in vivo

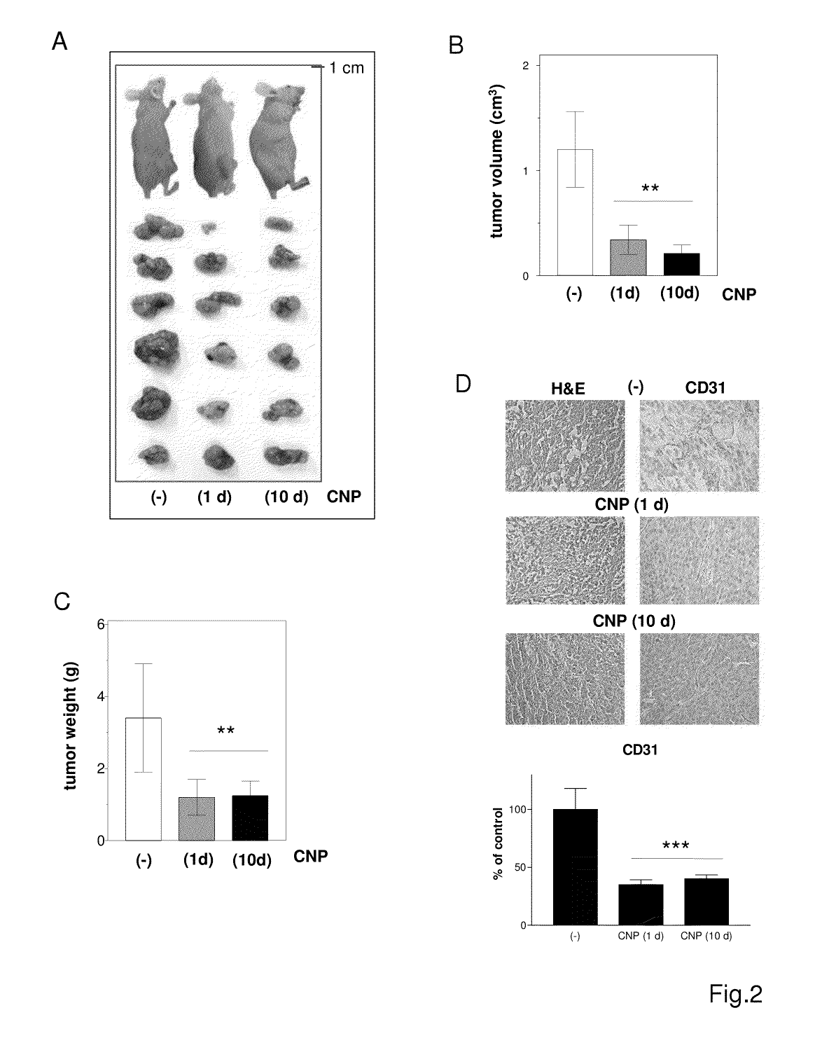

To investigate the effect of CNP on human malignant melanoma growth, A375 cells were injected subcutaneously to the flank of nude mice to initiate tumor development in vivo. Group 1 of six mice were untreated, group 2 was treated with CNP starting with day 1 post tumor cell injection and the last set (group 3) of tumor cell injected mice was treated with CNP starting with day 10 after injection. CNP was injected intraperitoneal every alternate day. After dissection of tumors from mice after 30 days post injection of tumor cells, a significant lowered tumor growth was observed for both applications of CNP (FIG. 2A). In contrast to mock-treated controls, a significant smaller tumor volume of about 75% (1 d) and 85% (10 d) was determined after CNP treatment (FIG. 2B). Furthermore, the tumor weight was significantly reduced for both CNP applications of about 70% compared with mock-treated tumor-bearing mice (FIG. 2C) indicating a positive effect of CNP on inhibition of malignant melanoma growth in vivo. At first view it seemed not clear, why there was no significant difference between a treatment with CNP starting at day 1 (1 d) or day 10 (10 d) post injection of tumor cells. However, it depended on the experimental approach and on a delay in the initial growth of the injected tumor cells. After a delay of growth in the order of 10 to 14 days showing some small nodules only, the growth of tumor cells not treated with CNP dramatically increased for the rest of the studied time period in contrast to CNP-treated tumor cells. Therefore, the treatment with CNP starting at 1 day or 10 days after tumor cell injection did not show a significant difference having a look on tumor volume or weight.

These data show for the first time in a in vivo model that CNP may have the potential as anticancer agent. In that context earlier published data show an alternate effect on stromal cells indicating the dual role of CNP, protective on stromal cells and damaging on tumor cells (2). As many studies implicate tumor-associated blood vessel formation as a central part in the process of growth, invasion and metastasis of malignancies (50), angiogenesis and tumor-associated neovascularization are complex processes by which new blood vessels are formed from pre-existing vasculature. In order to examine the effect of CNP on angiogenesis in vivo, each tumor was subjected to immunohistological analysis of CD31 (PECAM-1). CNP treatment starting with day 1 and day 10 after tumor cell injection in nude mice significantly lowered (˜60%) CD31 in the tumor sections compared with the control group (FIG. 2D). These data indicate an inhibiting effect of CNP on migration of endothelial cells in context to neovascularization which correlates with a lowered invasive capacity of CNP-treated endothelial cells in an in vitro matrigel assay (FIG. S2). The most widely used endothelial cell marker for studying angiogenesis/neovascularization is e.g. CD31 (PECAM-1). CD31 is strongly expressed by all endothelial cells and weakly expressed by several types of leukocytes (43). Staining of blood vessels with CD31 antibodies has been shown to be suitable for the identification of angiogenesis in several types of malignancies (23). According to the literature, the density and morphological state of microvessels in a tumor are closely correlated with prognosis and biological behavior. Therefore, CNP can be considered as promising antitumor agent.

On the basis of the promising results with the xenograft model, we performed a set of in vitro experiments to get insight into the underlying molecular mechanism modulated by CNP in melanoma.

### 3. Effect of Cerium Oxide Nanoparticles on Cell Proliferation

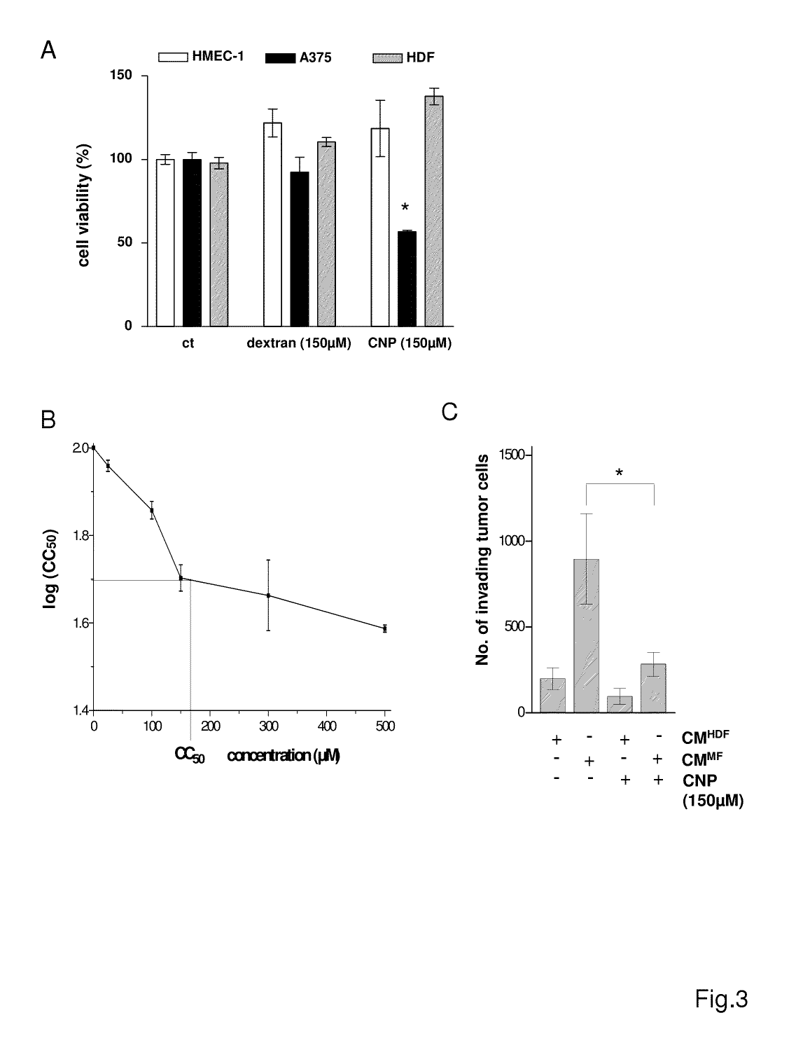

To examine a potential toxic effect of CNP, melanoma cells were incubated with 5 nm-sized nanoparticles for 96 h. The cell proliferation was analyzed by MTT assays. As shown in FIG. 3A, cell viability of A375 cells was decreased by treatment with 150 μM nanoceria for 96 h. A concentration of 150 μM CNP was used which was shown earlier (3) to be non-toxic for normal cells, but having a cytotoxic effect on squamous carcinoma cells. The cytotoxicity of polymer-coated cerium oxide nanoparticles on stromal cells (human dermal fibroblasts (HDF) and human endothelial cells (HMEC-1)) was compared with melanoma cells (FIG. 3A). The viability of normal cells was evidently not altered at 96 h after treatment with CNP, while melanoma cells showed a lowering of the viability by around 45%. However, no change of cell viability was observed for the three different cell types after treatment with dextran only (mock-treated) compared with untreated controls (ct). These results suggest that the cytotoxic effect of the dextran-coated nanoparticles depends on the redox active cerium oxide and is not affected by the polymer. That coated cerium oxide nanoparticles possess great selectivity between cancer and normal cells and it displays potential application in melanoma chemoprevention. In that context, the cytotoxic concentration values which cause destruction in 50% of the proliferating cells (CC₅₀) were determined being 166 μM for melanoma cells and no toxicity was observed for CNP concentrations up to 1 mM for fibroblasts and for the studied endothelial cells. That data indicate a higher sensitivity of melanoma cells against the polymer-coated nanoparticles (FIG. 3B).

### 4. Involvement of CNP in Tumor Invasion

As alpha-smooth muscle actin expressing myofibroblasts (MF), often found at the invasion front of many tumors (13) increase the invasive capacity of tumor cells (10), the potential of CNP to prevent invasion on melanoma cells was studied using the conditioned medium (CM) of myofibroblasts (CM^{MF}) and normal fibroblasts (CM^{HDF}), which were used as control. Therefore, A375 melanoma cells were incubated with different concentrations of CNP. Fourty-eight hours after treatment, the invasive capacity of these cells and mock-treated control cells was tested with CM^{HDF} and CM^{MF} (FIG. 3C). Indeed, the invasive capacity of tumor cells is modulated by CNP. Compared to mock-treated tumor cells, the invasive capacity of CNP-treated A375 cells was significantly lowered by 70% using both, CM^{HDF} and CM^{MF}.

### 5. Modulation of ROS Generation and Protein Oxidation by CNP

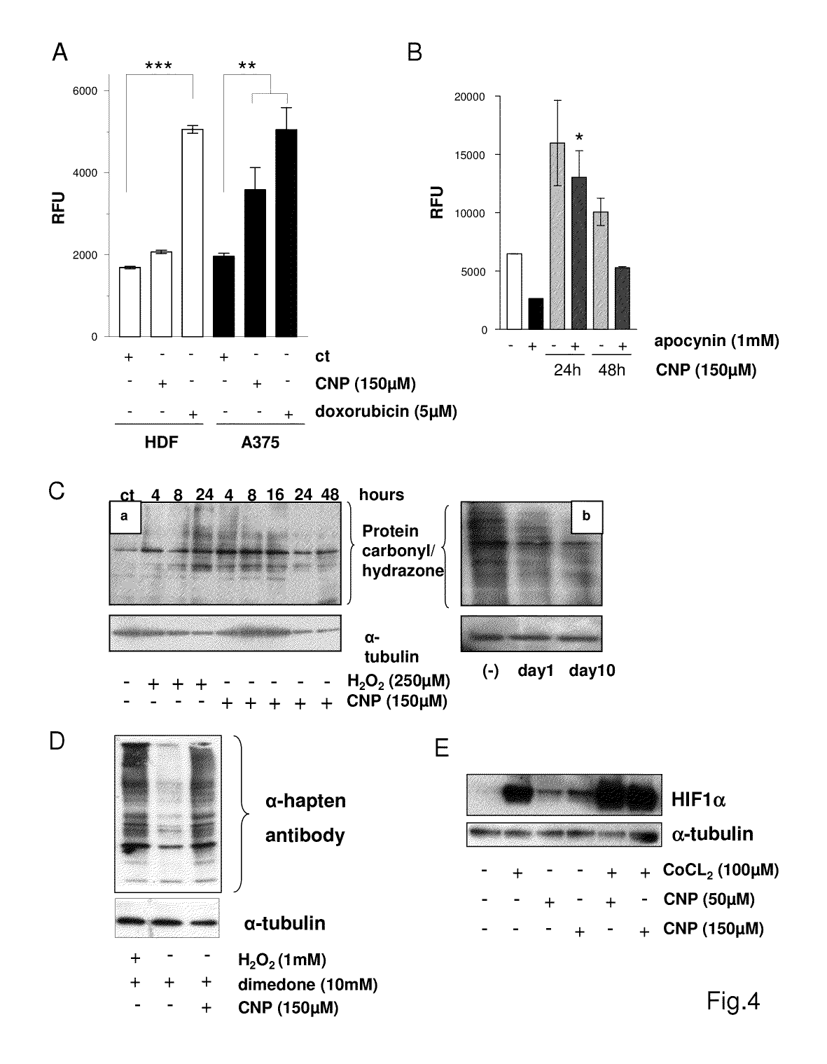

To study a possible effect of nanoceria-initiated ROS on toxicity and invasion of tumor cells, we assessed ROS generation both intracellularly and extracellularly. The intracellular ROS level of human dermal fibroblasts was compared with the ROS level of A375 melanoma cells. The treatment of both HDF and melanoma cells resulted in a significant 2-fold increase in the ROS level in the tumor cells whereas no increase was detected in the fibroblasts. Interestingly, the treatment of both cell types with the chemotherapeutical ROS-producing drug doxorubicin (14, 45), which was used as control, significantly increased the intracellular ROS level in both cell types (FIG. 4A). These data suggest a prooxidant effect of CNP in tumor cells and a different sensitivity of tumor cells against CNP compared with normal cells which could be a benefit of such nanoparticles in context of future therapeutical approaches. As CNP treatment resulted in an intracellular increase in ROS in tumor cells, a potential extracelluar increase in hydrogen peroxide should be measured with the chemically stable fluorogenic probe N-acetyl-3,7-dihydroxyphenoxazine (Amplex Red), which is highly sensitive for the determination of extracellular H202 (66). CNP exposure resulted in a significant increase in peroxide generation (FIG. 4B). A maximum of the H₂O₂ level was measured 24 h after treatment of A375 melanoma cells with CNP, which resulted in a 2.3-fold higher H₂O₂ amount compared to mocktreated controls. A decrease of the H₂O₂ amount was observed between 24-48 h. Melanoma cells treated for 48 h with CNP resulted in an up to 1.6 fold higher H₂O₂ level compared to mock-treated control. As the production of H₂O₂ by CNP needs a superoxide source (31), the effect of apocynin on the production of H₂O₂ was measured. Apocynin, a methoxy-substituted catechol, inhibits NAD(P)H oxidase by impeding the assembly of p47phox and p67phox subunits within the membrane NAD(P)H oxidase complex (54). Pre-incubation of melanoma cells for 1 h with apocynin prior to CNP treatment downregulated the CNP mediated hydrogen peroxide generation, both at 24 and 48 h. However, it is evident that CNP exposure results in a solid generation of hydrogen peroxide.

Another, more indirect approach to measure the intracellular generation of ROS, the occurence of carbonylated proteins, a biomarker for intracellular oxidative stress, was detected. For that, A375 melanoma cells were treated with CNP for 4-48 h and the carbonylated proteins verified. A low amount of carbonylated proteins was detected in mock-treated A375 cells whereas the amount was significantly increased in H₂O₂- and CNP-treated cells compared to mock-treated cells after all studied time points (FIG. 4C (a)). However, the highest amount of carbonylated proteins was detected at 4 h post treatment with CNP with a stepwise lowering of the amount between 8-48 h (FIG. 4C (a)). Furthermore, protein lysates prepared from flash frozen tissues were used in Western blot procedures to assess carbonylation of proteins in tumor of mice. A high amount of carbonylated proteins was detected in tumors of mice, which were not treated with CNP (FIG. 4C (b)). CNP treatment starting at day 1 and day 10 after tumor cell injection in nude mice and finished 30 days post injection lowered the amount of carbonylated proteins in the tumor lysates, compared to untreated mice. That lowered amount of oxidized proteins in CNP treated mice correlates with the cell culture data (FIG. 4C (a)) and is due to the time-dependent cytotoxic, cell killing effect of CNP. In other words, a decrease in the cell number results in a decrease of oxidized proteins. Even though the occurence of protein carbonyls offers a first hint for CNP-initiated oxidative stress, the measurement of those carbonyls is rather a general measure of an alteration of the cellular redox status. Therefore, a more specific approach, namely the detection of oxidized thiol (—SOH) groups by H₂O₂ (=sulfenic acids), was performed to measure a change in the intracellular redox status. The application of an α-hapten antibody directed against sulfenic acid of oxidized protein/peptide residues (51) showed a significant increase in oxidized thiols at 24 h after treatment with CNP compared to the diketone dimedone treated controls. In addition, treatment of the cells with exogenously added H₂O₂ resulted in a similarlevel of oxidized thiol groups (FIG. 4D).

A further parameter for testing a prooxidant status of cells is the accumulated translocation of the hypoxia inducible factor 1 alpha (HIF1α) to the nucleus (30) and the expression of HIF1α target genes, including proangiogenic factors and enzymes favoring a significant glycolytic metabolism (5, 48). Chemical inhibitors of the prolylhydroxylase, such as cobalt chloride, inhibit HIF-1α degradation and lead to its stabilization, therefore enhancing its detection (42). Treatment of A375 cells with a non-toxic concentration of 100 μM cobalt chloride for 4 h resulted in a significant stability of HIF1αprotein levels (FIG. 4E). Treatment with either 50 μM or 150 μM polymer-coated cerium oxide nanoparticles for 24 h led to an increase in HIF1α protein expression compared with untreated control. Co-incubation of CNP and CoCl₂ for 4 h showed an increased HIF1α protein level, which was significant higher than in the CoCl₂ treated controls.

As HIF-1α positively affects the expression of glycolytic enzymes resulting, for example, in a higher intracellular lactate and H+ production with a subsequent decrease of the microenvironmental pH of tumor cells (22, 57), the question was addressed of whether the prooxidant effect of CNP on tumor cells may due to a higher lactate/H+ production of these cells versus (stromal) fibroblasts. Indeed, the A375 melanoma cells produced up to 38% more lactate compared to the fibroblasts (FIG. S3). These increase in the lactate and H+ level explains the superoxide dismutase mimetic activity of nanoceria ((a) Ce⁴⁺+O₂^{.-}→Ce³⁺+0₂, (b) Ce³⁺+2H⁺+O₂^{.-}→Ce⁴⁺+H₂O₂), which was published earlier (3, 31).

The CNP mediated increase in the intracellular ROS level allows the conclusion that the change of the intracellular redox status in tumor cells may play an important role in the increased cellular toxicity and lowering of the invasive capacity. This hypothesis was checked in subsequent experiments.

### 6. Induction of Apoptosis by CNP

To assess whether the cytotoxic effect of polymer-coated cerium oxide nanoparticles in melanoma cells was caused by ROS-mediated apoptotic cell death, the occurrence of apoptotic biomarkers such as cytochrome c release, caspase-3 and Poly(ADP-ribose)polymerase (PARP) cleavage was measured.

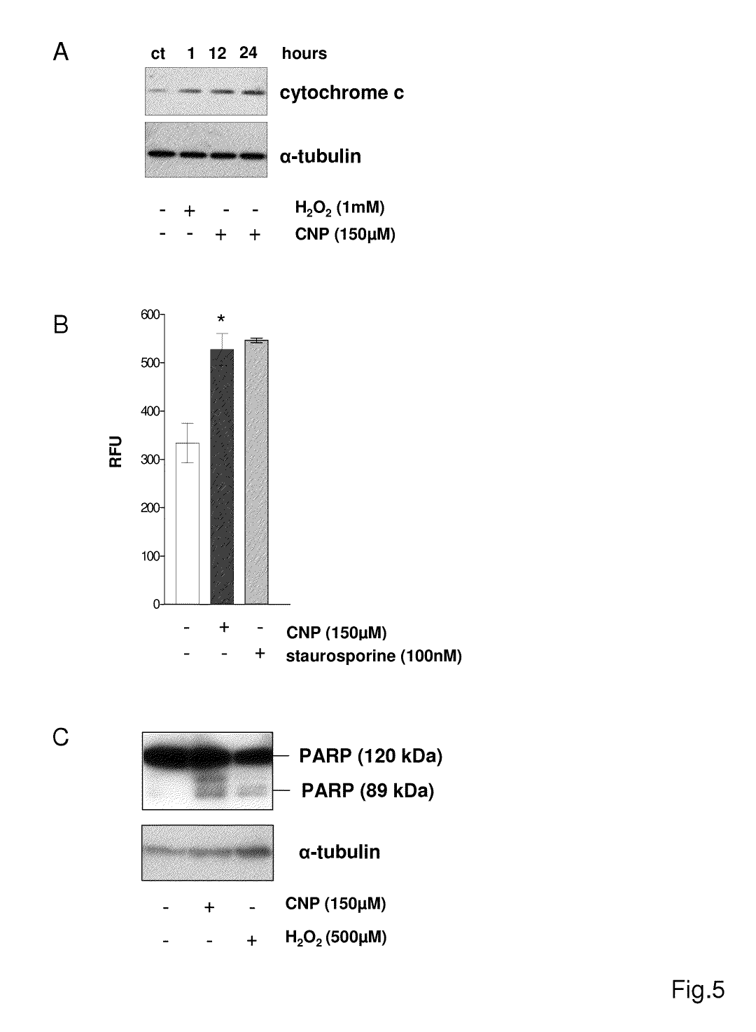

The release of cytochrome c is an early event of the intrinsic apoptotic cascade initiated, for example by ROS (29). In that context, treatment of the melanoma cells with H₂O₂ for 1 h resulted in a significant increase of cytosolic cytochrome c compared to mock-treated controls. A similar elevated level of cytosolic cytochrome c was detected after treatment of the tumor cells with CNP for 12 and 24 h (FIG. 5A).

The CNP treatment resulted in a 6.1 fold increase. As illustrated in FIG. 5B the activity of caspase-3 was significantly increased by CNP in A375 cells compared to mocktreated control cells. At 24 h post treatment with CNP an up to 1.9-fold increase in the caspase-3 activity was measured. Furthermore, the natural material staurosporine, which induces caspase-3 activity in melanoma cells (39) and which was used herein as positive control, increased the activity of caspase-3.

In addition to the cytochrome c release and caspase-3 activity assay, PARP cleavage, was evaluated. Caspase activation subsequently induces proteolytic cleavage of PARP, which serves as a biochemical marker of cells undergoing apoptosis (8). CNP treatment for 24 h induced PARP cleavage in melanoma cells compared to mock-treated control, which was demonstrated by the appearance of the 89 kDa-fragment (FIG. 5C). Treatment of the tumor cells with exogenously added H₂O₂ resulted in PARP cleavage as well. Apoptosis is initiated by two central mechanisms, the extrinsic (36) and intrinsic pathway (41). Exposure of A375 cells to CNP resulted in cytochome c release, activation of caspase-3 and PARP cleavage and (FIG. 5 A-C), which indicated the activation of the intrinsic pathway of apoptosis. In that context, it is described that gold nanoparticles (AuNPs) resulted in a significant increase in H₂O₂ generation and thus apoptosis is irreversibly induced (17). In conclusion, CNP lead to a prooxidant status of the tumor cells resulting in proapoptotic mechanism.

### 7. Caveolin-1 Downregulation by CNP

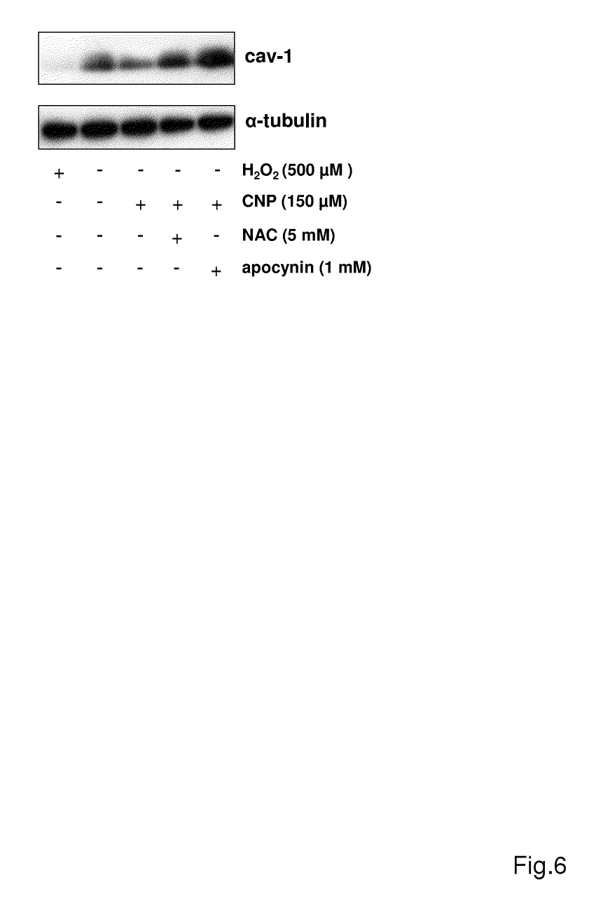

The role of reactive oxygen species in the cellular invasion and migration was described earlier (34, 55). Caveolin-1 (Cav-1) has received the most attention since its expression has been linked to cancer progression and aggressiveness (58). Cav-1 play a role in cell death and survival but also in cell migration (38) and invasion (60). It is consistent that both H₂O₂ and superoxide (O2^{.-}) have an inhibitory effect on expression of Cav-1, while hydroxyl radicals increase Cav-1 expression and migration (38). Melanoma cells were treated with CNP for 1 h and Cav-1 expression was detected. CNP caused a significant downregulation of Cav-1 expression compared to the mock-treated control (FIG. 6). Incubation of the cells with exogenous H₂O₂ for 1 h and 4 h completely abrogated caveolin-1 expression. By contrast, pretreatment of melanoma cells with the antioxidant N-acetyl-L-cysteine or apocynin resulted in an increased caveolin-1 expression compared to CNP treated cells. These data indicate the involvement of CNP-initiated ROS in lowering the invasive capacity of the studied tumor cells.

### Discussion of Examples

As both the incidence of melanoma is increasing faster than that of other cancers and the chemotherapeutical treatment of a majority of patients with metastatic melanoma often results in adverse reactions and response rates which are not high enough to significantly affect median survival, novel therapeutical approaches should be the objective for the near future. In this study, we have shown for the first time in vitro and in vivo, that concentrations of polymer-coated cerium oxide nanoparticles being non-toxic for stromal cells exhibit a direct reactive oxygen species-dependent cytotoxic (proapoptotic) and anti-invasive effect on melanoma cells. Our study highlights a prospective clinical significance of polymer-coated cerium oxide nanoparticles.

Recently, a number of studies have focused on the interaction between nanoparticles and biological systems in order to evaluate novel strategies for an efficient drug delivery and possible alternative cancer therapies. Magnetic iron nanoparticles e.g. are used in cancer therapy. Cancer cells incubated with this particles and then magnetically heated show decreased viability, whereas normal cells remain unaffected (32). Polymer-coated cerium oxide nanoparticles with a size of ˜20 nm and in higher concentrations then used herein generate free oxygen species in human lung cancer cells and produce significant oxidative stress and subsequent decrease in the cell viability (33). However, the effect of that high concentration of CNP on normal (stromal) cells was not studied. We described earlier, that CNP with a smaller size and lower concentration results in an antioxidant and protective effect in (stromal) fibroblasts (3). Cerium oxide nanoparticles (nanoceria) have a unique electronic structure which is mediated by their large surface-area-to-volume ratio (28) resulting in valence and oxygen defects on the surface of that particles. Therefore, nanoceria possess a promising pharmacological potential suggesting that nanoceria may be used as an antioxidant agent (11).

The antioxidant ability is associated with the superoxide dismutase (SOD) mimetic (21, 31) and catalase mimetic activities (44) of nanoceria which was measured primarily in a cell-free system. Recently, we published an antioxidative activity of cerium oxide nanoparticles in normal cells and, surprisingly, a prooxidant effect in cells of a squamous skin tumor (3). In that context, the data presented herein with melanoma cells support a prooxidant (SOD-mimetic) mechanism of nanoceria which depends on the pH of the cells. As a higher lactate/H⁺ production was detected in A375 cells (see FIG. S3) the following chemical mechanism is suggested: a) Ce⁴⁺+O₂^{.-}→Ce³⁺+O₂ and b) Ce³⁺+2H⁺+O₂^{.-}→Ce⁴⁺+H₂O₂. As FIGS. 4A and B indicate a production of superoxide and an increase in hydrogen peroxide in the melanoma cells and FIG. S1B shows a higher percentage of Ce⁴⁺ in coated nanoparticles, which is important for the initial SOD mimetic mechanism, these results in accordance with earlier published data support the proposed prooxidant effect of polymer-coated cerium oxide nanoparticles. That prooxidant effect results in cytotoxicity and decrease of the invasive capacity of the tumor cells.

### Materials and Methods

Cell culture medium (Dulbecco's modified Eagle's medium (DMEM) was purchased from Invitrogen (Karlsruhe, Germany) and the defined fetal calf serum (FCS gold) were from PAA Laboratories (Linz, Austria). All chemicals including protease as well as phosphatase inhibitor cocktail 1 and 2 were obtained from Sigma (Taufkirchen, Germany) or Merck Biosciences (Bad Soden, Germany) unless otherwise stated.

The protein assay kit (Bio-Rad DC, detergent compatible) was from BioRad Laboratories (München, Germany). Matrigel and polycarbonate cell culture inserts (6.5 mm diameter, 8 μm pore size) were delivered from BD Biosciences (Heidelberg, Germany). The Oxyblot Protein Oxidation Detection kit was from Millipore (Schwalbach, Germany), while the Amplex Red Hydrogen Peroxide/Peroxidase Assay Kit was provided from Invitrogen (Karlsruhe, Germany). The enhanced chemiluminescence system (SuperSignal West Pico/Femto Maximum Sensitivity Substrate) was supplied by Pierce (Bonn, Germany). Monoclonal mouse antibody raised against human αSMA and α-tubulin was supplied by Sigma. Polyclonal rabbit antibody raised against human HIF-1α and Caveolin-1 was supplied by New England Biolabs (Frankfurt a.M., Germany). The polyclonal rabbit α-hapten antibody directed against oxidized thiol groups (sulfenic acid) (50) was a gift from Kate S. Carrol's group. The following secondary antibodies were used: polyclonal horseradish peroxidase (HRP)—conjugated rabbit anti-mouse IgG antibody (DAKO, Glostrup, Denmark) and goat anti-rabbit immunoglobulin G antibodies were from Dianova

(Hamburg, Germany). Recombinant human TGFβ1 (rTGFβ1) was from R&D Systems (Wiesbaden, Germany).

### Cell Culture

Human dermal fibroblasts (HDF) were established by outgrowth from foreskin biopsies of healthy human donors with an age of 3-6 years. Cells were used in passages 2-12, corresponding to cumulative population doubling levels of 3-27 (6). The human malignant melanoma cell line A375, originally derived from a 54-year-old woman, was purchased from ATCC (20). The immortalized human microvascular endothelial cell line (HMEC-1), which expresses cell-surface molecules typically associated with primary endothelial cells, was obtained from the Centers for Disease Control (Atlanta, Ga., USA) (1). Dermal fibroblasts, melanoma and endothelial cells were cultured as described (56). Myofibroblasts (MF) were generated by treatment of HDFs with recombinant TGFβ1 (rTGFβ1) for 48 h in HDF conditioned medium (CM^{HDF}) (10).

### Preparation of Conditioned Medium

Conditioned medium was obtained from human dermal fibroblasts (CM^{HDF}) and myofibroblasts (CM^{MF}). For this, seeded 1.5×10⁶ HDF cells were grown to subconfluence (˜70% confluence) in 175-cm2 culture flasks. The serum-containing medium was removed, and after washing in phosphate-buffered saline (PBS) the cells were treated with 5 ng/ml or without rTGFβ1 in serum-free DMEM for 48 h. This medium was removed, and after washing in PBS all cells were incubated in 15 ml serum-free DMEM for further 48 h before collection of the conditioned medium of HDF (CM^{HDF}) and myofibroblasts (CM^{MF}). Conditioned media were freshly used or stored at −20° C. for at the most 2 weeks before use.

### Cell Viability

The cytotoxic effect of cerium oxide nanoparticles (CNP) was measured by MTT (3-(4,5-Dimethylthiazol-2-yl)-2,5-diphenyltetrazolium bromide) assay (40). The activity of mitochondrial dehydrogenases, as indicator of cellular viability, results in the formation of a purple formazan dye. Briefly, MTT solution (0.5 mg/ml) was added to the cells treated with different concentrations of CNP for 96 h after washing with PBS. The medium was removed and the cells were lysed in dimethyl sulfoxide. The formazan formation was measured at 570 nm. The results were presented as percentage of mock-treated control which was set at 100%. The CC₅₀ value was defined as the concentration which reduced the absorbance of treated cells to 50% when compared to mock-treated (control) cells.

### Synthesis of Cerium Oxide Nanoparticles

Cerium oxide nanoparticles were synthesized in dextran (molecular weight: 1000 Da) using previously described methods (26). Briefly, cerium nitrate hexahydrate was dissolved in deionized water and the pH of the solution was maintained between 3.5 and 4.0 for uncoated (water-based) nanoparticles. Stoichiometric amounts of hydrogen peroxide and ammonium hydroxide were added to oxidize the dissolved cerium ions as cerium oxide nanoparticles. The pH of the solution needs to be maintained strictly below 4.0 to avoid precipitation of the nanoparticles. For synthesis of dextran coated nanoparticles a stoichiometric amount of dextran (5 mM) was at first dissolved in deionized water (18.2 MΩ) followed by cerium nitrate hexahydrate. The solution was stirred for 2 h followed by addition of ammonium hydroxide (30% w/w) to oxidize the dissolved cerium ions as cerium oxide nanoparticles. The pH of the solution was kept below 9.5 to avoid precipitation of cerium hydroxide. The resulting dextran-coated cerium oxide nanoparticles (CNP, nanoceria) were analyzed using UV-Visible spectroscopy for determining the oxidation state of nanoparticles. Transmission electron microscopy and dynamic light scattering were used to determine the particle size and hydrodynamic radius of the nanoparticles. Catalse and superoxide dismutase (SOD) activity were determined as described in our previous publication (3). After preparation of dex-CNP, we dialyzed the solution to remove excess dextran for 48 h against dH2O using 1200 molecular wt cut off dialysis membrane, and then FTIR was carried out to confirm coating/presence of dextran on the surface of CNP. FTIR data of bare CNP, Dextran and Dextran-CNP is shown in FIG. S5. FTIR data confirms presence of dextran coating on the surface of the CNP. Fourier transform infrared (FTIR) spectra were collected to confirm the Dextran molecule on the nanoparticle surface using PerkinElmer Lamda750S and PerkinElmer Spectrum.

### High Resolution Transmission Electron Microscopy (HRTEM)

High resolution transmission electron micrographs were obtained using FEI Tecnai F 30 microscope operated at 300 kV with a point-to-point resolution of 0.2 nm. The samples were prepared by depositing a drop of dextran-coated CNP on a holy carbon coated copper grid. The grids were dried overnight in vacuum before imaging.

### X-ray Spectroscopy

X-Ray photoelectron spectroscope (XPS) spectrum of Ce(3d) were collected using 5400 PHI ESCA (XPS) spectrometer and Mg-Kα X-radiation (1253.6 eV) at a power of 350 watts was used during the data collection. Ce^{4+/}Ce³⁺ oxidation states ratio on the surafce of bare and dextran coated cerium oxide nanoperticles were analyzed by deconvoluting the X-Ray photoelectron spectroscope (XPS) spectrum of Ce(3d) as described elase where (12). The peaks at 880.8, 885.8, 899.3 and 903.5 eV corresponding to the Ce³⁺ oxidation state and peaks at, 881.9, 888.4, 897.9, 901.2, 906.8 and 916.3 eV corresponding to the Ce⁴⁺ were assigned. Intensities of the peaks were determined and Ce^{3+/}Ce⁴⁺ ratios calculated.

### Cellular Uptake of Nanoparticles

Human melanoma cells in serum-free Dulbecco's Modified Eagle Media (DMEM) were treated with 150 μM CNP for 4 h. Thereafter, cells were harvested and washed with phosphate-buffered saline (PBS) to remove excess media. As CNP is not detectable by phase contrast microscopy, transmission electron microscopy was used to determine the cellular uptake of nanoceria. For electron microscopy, pelleted samples of CNP treated cells were fixed for 2 h in 4% paraformaldehyde and 2.5% glutaraldehyde (Serva, Heidelberg, Germany) in 0.1 M phosphate buffer at pH 7.4 at room temperature. Next, the pellets were thoroughly washed with four changes of PBS, followed by a postfixation for 60 min in 1% osmium tetroxide (Serva) in PBS. The specimens were dehydrated in a graded series of acetone, and embedded in Spurr's medium (Serva) at 70° C. for 24 h. Ultrathin sections were cut from the embedded tissue with a Reichert Ultracut (Vienna, Austria) using a diamond knife. The sections were collected on coated copper grids, and subsequently stained with uranyl acetate and lead citrate according to earlier published data (46). The grids were analyzed using a Hitachi H 600 electron microscope. Documentation was carried out by using an optical system and the Digital Micrograph software (Gatan, Munich, Germany). For light microscopical controls semithin section were cut and stained with 1% Toluidine blue and 1% Borax. For fluorescence microscopy, cells were incubated with 150 μM FITC-labelled CNP for 4 h or mock-treated. Thereafter, cells were washed twice with PBS and fixed with methanol. For preparation ProLong Gold (Invitrogen) was used, a reagent that simultaneously stains the nuclei with DAPI. The fluorescence microscopic examination was done with a Zeiss Axiovert 100TV and the documentation with a digital camera system (Hamamatsu C4742-95).

### SDS-PAGE and Western Blotting

SDS-PAGE was performed according to the standard protocols published elsewhere (33) with minor modifications. Briefly, cells were lysed after incubation with CNP in 1% SDS with 1:1000 protease inhibitor cocktail (Sigma; Taufkirchen, Germany). After sonication, the protein concentration was determined by using a modified Lowry method (Bio-Rad DC). 4×SDS-PAGE sample buffer (1.5M Tris-HCl pH 6.8, 6 ml 20% SDS, 30 ml glycerol, 15 ml β-mercaptoethanol and 1.8 mg bromophenol blue) was added, and after heating, the samples (20-3 μg total protein/lane) were applied to 8-15% (w/v) SDS-polyacrylamide gels. After electroblotting, immunodetection was carried out (1:1000 dilution of primary antibodies rabbit monoclonal anti αSMA, HIF1α, Caveolin-1 and mouse monoclonal anti α-tubulin), 1:20000 dilution of antimouse/rabbit antibody conjugated to HRP). Antigen-antibody complexes were visualized by an enhanced chemiluminescence system. α-tubulin was used as internal control for equal loading.

### A375 Xenograft Model of Nude Mice

One million human melanoma A375 cells (1×10⁶) were resuspended in 50 μl phosphate-buffered saline (PBS), mixed with 2000 Matrigel (BD Biosciences, San Jose Calif.), and implanted subcutaneously into the right flank of 6-7 weeks old athymic NCr-nu/nu mice from National Cancer Institute (NCI), Frederick, USA. The care and experimental manipulation of mice described in this study was in accordance with guidelines of the Mayo Clinic College of

Medicine for the ethical treatment of animals. In order to distinguish between individual differences in mice, six mice were chosen for each group. Group 1 is vehicle treated (=mock-treated (0.1 mg dextran/kg body weight)); group 2 is CNP treated starting with day 1 after tumor cells were injected; and group 3 is CNP treated starting with day 10 post injection of tumor cells. Nanoceria were administered by intraperitoneal injection, every alternate day with the dose of 0.1 mg/kg body weight for 30 days. The day after the last CNP application, the tumor was dissected and its weight recorded. Three diameters of the tumor (L, B, and H) were measured by caliper. The volume of the tumor was calculated as V=π/6×L×B×H (62).

### Immunohistochemistry/Histology

Tumor angiogenesis was assessed by CD31/PECAM-1 (Platelet Endothelial Cell Adhesion Molecule-1) staining of paraffin tissue sections employing a rat monoclonal antibody (Dianova, Hamburg, Germany), a biotinylated secondary antibody (Biotin Goat anti-Rat Ig, BD Pharmingen, Franklin Lakes, N.J., USA) and streptavidin/peroxidase and diaminobenzidine/hydrogen peroxide (Dako, Glostrup, Denmark). CD31 staining was quantitated by image analysis using graphpad prsm software.

### Measurement of Extracellular and Intracellular ROS

The generation of ROS was determined using the Amplex Red Hydrogen Peroxide/Peroxidase Assay Kit. The Amplex Red reagent (10-acetyl-3,7-dihydroxyphenoaxine), in combination with horseradish peroxidase (HRP), can be used to detect hydrogen peroxide released from cells. H₂O₂-mediated oxidation of the reagent results in the fluorescent product resorufin. Subconfluent A375 tumor cells were treated with 150 μM CNP or pre-incubated with 1 mM apocynin in serumfree DMEM in 96-well plates for different time points. Untreated subconfluent A375 cells were used as negative controls. After incubation with CNP, cells were washed twice with Hanks Balanced Salt Solution (HBSS) and the assay was performed following the manufacturer's instruction. Resorufin fluorescence was detected at an excitation wavelength of 520 nm and emission wavelength of 590 nm in a FLUOstar OPTIMA plate reader (BMG Labtech, Offenburg, Germany). Mean fluorescence intensities and standard error of mean were determined by using the statistical software Prism 3.0 (GraphPad, San Diego, Calif, USA). Furthermore, the generation of intracellular ROS was determined using 2?,7?-Dichlorodihydrofluorescein diacetate (H2DCF-DA), a dye that diffuses across the lipid membranes into cells and is subsequently oxidized by intracellular ROS forming the highly fluorescent DCF.

Untreated subconfluent human dermal fibroblasts (HDF) and melanoma cells (A375) were used as negative controls. Medium was substituted after 24 h starvation by 100 μM H2DCF-DA containing Hanks Balanced Salt Solution (HBSS) for 30 minutes. Subconfluent HDF and A375 tumor cells were exposed to 150 μM CNP and 5 μM doxorubicin in serum-free DMEM in 24-well plates and DCF fluorescence was detected at an excitation wavelength of 485 nm and emission wavelength of 520 nm in 5 minutes intervals for 90 minutes in a FLUOstar OPTIMA plate reader (BMG Labtech, Offenburg, Germany). Mean fluorescence intensities and standard error of mean were determined for each reading point by using the statistical software Prism 3.0 (GraphPad, San Diego, Calif., USA) and the end point is shown.

### Determination of Oxidized (Carbonylated) Proteins

A375 melanoma cells were grown to subconfluence on tissue culture dishes. After removal of serum-containing medium, cells were cultured in serum-free medium and either mock-treated or treated for different timeperiods with 150 μM CeO₂ nanoparticles. As positive control, the cells were treated with 250 μM H₂O₂. Thereafter, cells were lysed and carbonyl groups of oxidized proteins were detected with the OxyBlot™ Protein Oxidation Detection Kit, following the manufacturer's protocol. Briefly, the protein concentration was determined by using a modified Lowry method (Bio-Rad DC). Five μg of the cell lysates were incubated with 2,4-dinitrophenyl (DNP) hydrazine to form the DNP hydrazone derivatives. Labeled proteins were separated by SDS-PAGE and immunostained using rabbit anti-DNP antiserum (1:500) and goat anti-rabbit IgG conjugated to horseradish peroxidase (1:2000). Blots were developed by enhanced chemiluminescence.

### Determination of Oxidized Thiol Groups (Sulfenic Acid)

A375 melanoma cells were grown to subconfluence on tissue culture dishes. After removal of serum-containing medium, cells were cultured in serum free medium and either mock-treated or treated for 24 h 150 μM CeO₂ nanoparticles and the last 2 h with 10 mM dimedone. As positive control, the cells were co-incubated with 10 mM dimedone and 1 mM H₂O₂ for 2 h. Cells were harvested, washed with PBS and lysates were generated and analyzed with the α-hapten (1:1000) antibody directed against oxidized SH-groups by western blot analysis.

### Invasion Assay

Cell culture inserts (transwells) were overlaid with 125 μm/ml growth factor reduced Matrigel and placed in a 24-well plate. A375 tumor cells (5×10⁴ cells/insert) either mock-treated or pretreated with 150 μM CNP were seeded on top of the matrigel in serum-free DMEM. CMHDF and CMMF (see above) were used as chemoattractant in the lower chamber. After 30 h at 37° C., the melanoma cells were rubbed off the upper side of the filter using cotton swabs, and the A375 cells, which invaded towards the lower side of the insert, were stained with Coomassie Blue solution (0.05% Coomassie Blue, 20% MeOH, 7.5% acetic acid). The number of invaded cells was estimated by counting random microscopic fields/insert.

### L-Iactate Assay

A375 melanoma cells and human dermal fibroblasts (HDF) were grown to subconfluence on tissue culture dishes. After removal of serum-containing medium, cells were cultured in serum-free medium for 24 h. Thereafter, cells were collected by centrifugation (1500 xg for 10 min at 4° C.). The supernatant was used to quantify extracellular I-lactate with the L-Lactate Assay Kit from Cayman Chemical Company following the manufacturer's protocol.

### Caspase-3 Activity Assay

A375 melanoma cells and human dermal fibroblasts (HDF) were grown to subconfluence on tissue culture dishes. After removal of serum-containing medium, cells were cultured in serum-free medium with 150 μM CNP or 100 nM staurosporine for 24 h. Cell pellets were suspended in cell lysis buffer and incubated on ice for 45 min. The lysate was vortexed every 15 min. After centrifugation at 11000×g for 15 min, supernatants were collected and immediately measured for protein concentration and caspase activity. Briefly, cell lysates (20 μg protein) were placed in 96-well plates and then the specific caspase substrate (Ac-DEVD-AMC for caspase-3) was added. Plates were incubated at 37° C. for 1 h and caspase activity was determined by fluorescence intensity with the excitation and emission wavelengths set at 380 nm and 440 nm, respectively.

### Statistical Analysis

Means were calculated from at least three independent experiments, and error bars represent standard error of the mean (s.e.m.). Analysis of statistical significance was performed by Student t test or ANOVA with *P<0.05, **P<0.01, and ***P<0.001 as levels of significance.

### REFERENCES

1. Ades E W, Candal F J, Swerlick R A, George V G, Summers S, Bosse D C, and Lawley T J. HMEC-1: establishment of an immortalized human microvascular endothelial cell line. J Invest Dermatol 99: 683-90, 1992.

2. Alili L, Karakoti A S, Seal S, and Brenneisen P. Suppression of tumor invasion by inorganic nanoparticles Cancer Research 69(23 Suppl.): Boston, C42, 2009.

3. Alili L, Sack M, Karakoti A S, Teuber S, Puschmann K, Hirst S M, Reilly C M, Zanger K, Stahl W, Das S, Seal S, and Brenneisen P. Combined cytotoxic and anti-invasive properties of redox-active nanoparticles in tumor-stroma interactions. Biomaterials 32: 2918-29, 2011.

4. Altekruse S F, K C, Krapcho M, Neyman N, Aminou R, Waldron W, Ruhl J, Howlader N, Tatalovich Z, Cho H, Mariotto A, Eisner M P, Lewis D R, Cronin K, Chen H S, Feuer E J, Stinchcomb D G, Edwards B K, SEER Cancer Statistics Review, 1975-2007. National Cancer Institute. Bethesda, MD, 2010.

5. Bardos J I, and Ashcroft M. Negative and positive regulation of HIF-1: a complex network. Biochim Biophys Acta 1755: 107-20, 2005.

6. Bayreuther K, Francz P I, Gogol J, and Kontermann K. Terminal differentiation, aging, apoptosis, and spontaneous transformation in fibroblast stem cell systems in vivo and in vitro. Ann N Y Acad Sci 663: 167-79, 1992.

7. Bhatia S, Tykodi S S, and Thompson J A. Treatment of metastatic melanoma: an overview. Oncology (Williston Park) 23: 488-96, 2009.

8. Boulares A H, Yakovlev A G, Ivanova V, Stoica B A, Wang G, Iyer S, and Smulson M. Role of poly(ADP-ribose) polymerase (PARP) cleavage in apoptosis. Caspase 3-resistant PARP mutant increases rates of apoptosis in transfected cells. J Biol Chem 274: 22932-40, 1999.

9. Brenneisen P, Wenk J, Klotz L O, Wlaschek M, Briviba K, Krieg T, Sies H, and Scharffetter-Kochanek K. Central role of Ferrous/Ferric iron in the ultraviolet B irradiation-mediated signaling pathway leading to increased interstitial collagenase (matrix-degrading metalloprotease (MMP)-1) and stromelysin-1 (MMP-3) mRNA levels in cultured human dermal fibroblasts. J Biol Chem 273: 5279-87, 1998.

10. Cat B, Stuhlmann D, Steinbrenner H, Alili L, Holtkotter O, Sies H, and Brenneisen P. Enhancement of tumor invasion depends on transdifferentiation of skin fibroblasts mediated by reactive oxygen species. Journal of Cell Science 119: 2727-2738, 2006.

11. Celardo I, Traversa E, and Ghibelli. Cerium oxide nanoparticles: a promise for applications in therapy. J Exp Ther Oncol 9: 47-51, 2011.

12. Deshpande S, Patil S, Kuchibhatla, S V, and Seal S. Size dependency variation in lattice parameter and valency states in nanocrystalline cerium oxide. Appl. Phys. Lett. 87, 133113, 2005.

13. De Wever O, and Mareel M. Role of myofibroblasts at the invasion front. Biological Chemistry 383: 55-67, 2002.

14. Fang J, Nakamura H, Iyer A K. Tumor-targeted induction of oxystress for cancer therapy. J Drug Target. 15: 475-86, 2007.

15. Freitas R A Jr. What is nanomedicine? Nanomedicine 1: 2-9, 2005.

16. Fruehauf J P, and Trapp V. Reactive oxygen species: an Achilles' heel of melanoma? Expert Rev Anticancer Ther 8: 1751-7, 2008.

17. Gao W, Xu K, Ji L, and Tang B. Effect of gold nanoparticles on glutathione depletion-induced hydrogen peroxide generation and apoptosis in HL7702 cells. Toxicol Lett 205: 86-95, 2011.

18. Garbe C, and Leiter U. Melanoma epidemiology and trends. Clin Dermatol 27: 3-9, 2009.

19. Garbe C, Terheyden P, Keilholz U, Kolbl O, and Hauschild A. Treatment of melanoma. Dtsch Arztebl Int 105: 845-51, 2008.

20. Giard D J, Aaronson S A, Todaro G J, Arnstein P, Kersey J H, Dosik H, and Parks W P. In vitro cultivation of human tumors: establishment of cell lines derived from a series of solid tumors. J Natl Cancer Inst 51: 1417-23, 1973.

21. Heckert E G, Karakoti A S, Seal S, and Self W T. The role of cerium redox state in the SOD mimetic activity of nanoceria. Biomaterials 29: 2705-9, 2008.

22. Helmlinger G, Yuan F, Dellian M, and Jain R K. Interstitial pH and pO2 gradients in solid tumors in vivo: high-resolution measurements reveal a lack of correlation. Nat Med 3: 177-82, 1997.

23. Jahroudi N, and Greenberger J S. The role of endothelial cells in tumor invasion and metastasis. J Neurooncol 23: 99-108, 1995.

24. Jemal A, Siegel R, Ward E, Hao Y, Xu J, Murray T, and Thun M J. Cancer statistics, 2008. CA Cancer J Clin 58: 71-96, 2008.

25. Kappus H, and Sies H. Toxic drug effects associated with oxygen metabolism: redox cycling and lipid peroxidation. Experientia 37: 1233-41, 1981.

26. Karakoti A S, S V N, Kuchibhatla T, Babu K S, and Seal S. Direct synthesis of nanoceria in aqueous polyhydroxyl solutions. Journal of Physical Chemistry C 111: 17232-17240, 2007.

27. Karakoti A S, Monteiro-Riviere N A, Aggarwal R, Davis J P, Narayan R J, Self W T, McGinnis J, and Seal S. Nanoceria as Antioxidant: Synthesis and Biomedical Applications. Jom (1989) 60: 33-37, 2008.

28. Karakoti A S, Singh S, Dowding J M, Seal S, and Self W T. Redox-active radical scavenging nanomaterials. Chem Soc Rev. 39: 4422-32, 2010.

29. Kawiak A, Zawacka-Pankau J, Wasilewska A, Stasilojc G, Bigda J, and Lojkowska E Induction of Apoptosis in HL-60 Cells through the ROS-Mediated Mitochondrial Pathway by Ramentaceone from Drosera aliciae. Journal of Natural Products 75: 9-14, 2012.

30. Kietzmann T, and Gorlach A. Reactive oxygen species in the control of hypoxia-inducible factor-mediated gene expression. Semin Cell Dev Biol 16: 474-86, 2005.

31. Korsvik C, Patil S, Seal S, and Self W T. Superoxide dismutase mimetic properties exhibited by vacancy engineered ceria nanoparticles. Chem Commun (Camb): 1056-8, 2007.

32. Kuchibhatla S, Karakoti A S, Bera D and Seal S. One dimensional nanostructured materials. Progress in Materials Science 52: 699-913, 2007.

33. Laemmli U K. Cleavage of structural proteins during the assembly of the head of bacteriophage T4. Nature 227: 680-5, 1970.

34. Laurent A, Nicco C, Chereau C, Goulvestre C, Alexandre J, Alves A, Levy E, Goldwasser F, Panis Y, Soubrane O, Weill B, and Batteux F. Controlling tumor growth by modulating endogenous production of reactive oxygen species. Cancer Res 65: 948-56, 2005.

35. Levi F, Boffetta P, and La Vecchia C. High constant incidence rates of second primary neoplasms. Eur J Cancer Prey 17: 385-8, 2008.

36. Li P, Nijhawan D, Budihardjo I, Srinivasula S M, Ahmad M, Alnemri E S, and Wang X. Cytochrome c and dATP-dependent formation of Apaf-1/caspase-9 complex initiates an apoptotic protease cascade. Cell 91: 479-89, 1997.

37. Lin W, Huang Y W, Zhou X D, and Ma Y. Toxicity of cerium oxide nanoparticles in human lung cancer cells. Int J Toxicol 25: 451-7, 2006.

38. Luanpitpong S, Talbott S J, Rojanasakul Y, Nimmannit U, Pongrakhananon V, Wang L, and Chanvorachote P. Regulation of lung cancer cell migration and invasion by reactive oxygen species and caveolin-1. J Biol Chem 285: 38832-40, 2010.

39. Mafecki J M, Bentke A, Ostrowska B, and Laidler P. Cytochalasin D, LY294002 and olomoucine synergize in promoting death of melanoma cells through activation of caspase-3 and apoptosis. Melanoma Res. 20: 52-8, 2010.

40. Mosmann T. Rapid colorimetric assay for cellular growth and survival: application to proliferation and cytotoxicity assays. J Immunol Methods 65: 55-63, 1983.

41. Nagata S. Apoptosis by death factor. Cell 88: 355-65, 1997.

42. Nangaku M, Izuhara Y, Takizawa S, Yamashita T, Fujii-Kuriyama Y, Ohneda O, Yamamoto M, van Ypersele de Strihou C, Hirayama N, and Miyata T. A novel class of prolyl hydroxylase inhibitors induces angiogenesis and exerts organ protection against ischemia. Arterioscler Thromb Vasc Biol 27: 2548-54, 2007.

43. Parums D V, Cordell J L, Micklem K, Heryet A R, Gatter K C, and Mason D Y. JC70: a new monoclonal antibody that detects vascular endothelium associated antigen on routinely processed tissue sections. J Clin Pathol 43: 752-7, 1990.

44. Pirmohamed T, Dowding J M, Singh S, Wasserman B, Heckert E, Karakoti A S, King J E S, Seal S, and Self W T. Nanoceria exhibit redox state-dependent catalase mimetic activity. Chem. Commun. 46: 2736-273, 2010.

45. Quiles J L, Huertas J R, Battino M, Mataix J, and Ramirez-Tortosa M C. Antioxidant nutrients and adriamycin toxicity. Toxicology 180: 79-95, 2002.

46. Reynolds E S. Use of Lead Citrate at High Ph as an Electron-Opaque Stain in Electron Microscopy. Journal of Cell Biology 17: 208, 1963.

47. Roberts R A, Smith R A, Safe S, Szabo C, Tjalkens R B, and Robertson F M. Toxicological and pathophysiological roles of reactive oxygen and nitrogen species. Toxicology 276: 85-94, 2010.

48. Ruas J L, and Poellinger L. Hypoxia-dependent activation of HIF into a transcriptional regulator. Semin Cell Dev Biol 16: 514-22, 2005.

49. Sanchez Y, Amran D, de Blas E, and Aller P. Regulation of genistein-induced differentiation in human acute myeloid leukaemia cells (HL60, NB4) Protein kinase modulation and reactive oxygen species generation. Biochemical Pharmacology 77: 384-396, 2009.

50. Saphir A. Angiogenesis: the unifying concept in cancer? J Natl Cancer Inst 89: 1658-9, 1997.

51. Seo Y H, and Carroll K S. Profiling protein thiol oxidation in tumor cells using sulfenic acid-specific antibodies. Proceedings of the National Academy of Sciences of the United States of America 106: 16163-16168, 2009.

52. Sies H. Strategies of Antioxidant Defense. European Journal of Biochemistry 215: 213-219, 1993.

53. Sies H, and Cadenas E. Oxidative Stress—Damage to Intact-Cells and Organs. Philosophical Transactions of the Royal Society of London Series B-Biological Sciences 311: 617-631, 1985.

54. Stolk J, Hiltermann T J, Dijkman J H, and Verhoeven A J. Characteristics of the inhibition of NADPH oxidase activation in neutrophils by apocynin, a methoxysubstituted catechol. Am J Respir Cell Mol Biol 11: 95-102, 1994.

55. Storz P. Reactive oxygen species in tumor progression. Front Biosci 10: 1881-96, 2005.

56. Stuhlmann D, Ale-Agha N, Reinehr R, Steinbrenner H, Ramos M C, Sies H, and Brenneisen P. Modulation of homologous gap junctional intercellular communication of human dermal fibroblasts via a paracrine factor(s) generated by squamous tumor cells. Carcinogenesis 24: 1737-48, 2003.

57. Swietach P, Vaughan-Jones R D and Harris A L. Regulation of tumor pH and the role of carbonic anhydrase 9. Cancer Metastasis Rev 26: 299-310, 2007.

58. Tang Y, Zeng X, He F, Liao Y, Qian N, and Toi M. Caveolin-1 is related to invasion, survival, and poor prognosis in hepatocellular cancer. Med Oncol, 29: 977-84, 2012.

59. Thannickal V J, Aldweib K D L, and Fanburg B L. Tyrosine phosphorylation regulates H2O2 production in lung fibroblasts stimulated by transforming growth factor beta 1. Journal of Biological Chemistry 273: 23611-23615, 1998.

60. Thannickal V J, and Fanburg B L. Reactive oxygen species in cell signaling. American Journal of Physiology-Lung Cellular and Molecular Physiology 279: L1005-L1028, 2000.

61. Thompson T C, Tahir S A, Li L, Watanabe M, Naruishi K, Yang G, Kadmon D, Logothetis C J, Troncoso P, Ren C, Goltsov A, and Park S. The role of caveolin-1 in prostate cancer: clinical implications. Prostate Cancer Prostatic Dis 13: 6-11, 2010.

62. Tomayko M M, and Reynolds C P. Determination of Subcutaneous Tumor Size in Athymic (Nude) Mice. Cancer Chemotherapy and Pharmacology 24: 148-154, 1989.

63. Valko M, Leibfritz D, Moncol J, Cronin M T, Mazur M, and Telser J. Free radicals and antioxidants in normal physiological functions and human disease. Int J Biochem Cell Biol 39: 44-84, 2007.

64. Wittgen H G, and van Kempen L C. Reactive oxygen species in melanoma and its therapeutic implications. Melanoma Res 17: 400-9, 2007.

65. Woiniak A, Drewa G, Wozniak B, Schachtschabel D O, Mila-Kierzenkowska C, Drewa T, Olszewska-Slonina D, and Soponska M. The effect of antitumor drugs on oxidative stress in B16 and S91 melanoma cells in vitro. Med Sci Monit 11: BR22-9, 2005.

66. Zhou M, Diwu Z, Panchuk-Voloshina N, and Haugland R P. A stable nonfluorescent derivative of resorufin for the fluorometric determination of trace hydrogen peroxide: applications in detecting the activity of phagocyte NADPH oxidase and other oxidases. Anal Biochem 253:162-8, 1997.

It should be borne in mind that all patents, patent applications, patent publications, technical publications, scientific publications, and other references referenced herein and in the accompanying appendices are hereby incorporated by reference in this application to the extent not inconsistent with the teachings herein.

It is important to an understanding to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A composition for treating skin cancer comprising polymer-coated cerium oxide nanoparticles, wherein said nanoparticles possess a coating that selectively produces oxidative stress in skin cancer cells.

2. The composition of claim 1, wherein said nanoparticles are of a size between 1-10 nm.

3. The composition of claim 1, wherein said nanoparticles selectively increase H₂O₂ in skin cancer cells compared to non-cancer cells.

4. The composition of claim 1, wherein said skin cancer cells are melanoma cells.

5. A method of treating skin cancer in a subject in need, said method comprising administering to said subject a therapeutically effective amount of a composition comprising polymer-coated cerium oxide nanoparticles.

6. The method of claim 5, wherein said skin cancer is melanoma.

7. The method of claim 5, wherein said nanoparticles are of a size between 1-10 nm.

8. The method of claim 5, wherein said nanoparticles are comprised of cerium-oxide.

9. The method of claim 5, wherein said nanoparticles are coated with Dextran.

10. The method of claim 5, wherein said nanoparticles are comprised of cerium-oxide coated with dextran.

11. The method of claim 5, wherein said composition is administered topically, parenterally, or orally.

12. The method of claim 5, wherein said composition is administered topically.

13. A composition for treating skin cancer comprising polymer-coated cerium oxide nanoparticles, wherein said nanoparticles possess a coating that selectively produces oxidative stress in skin cancer cells; and wherein said polymer is dextran.

14. A method of treating skin cancer in a subject in need, said method comprising administering to said subject a therapeutically effective amount of a composition according to claim 13.
